(19) United States
(12) Patent Application Publication
(10) Pub. No.: US 2023/0183266 A1
(43) Pub. Date: Jun. 15, 2023

(54) CRYSTALLINE FORMS OF RET INHIBITOR AND PREPARATION THEREOF

(71) Applicant: LOXO ONCOLOGY, INC., Indianapolis, IN (US)

(72) Inventors: Jeremy Miles MERRITT, Indianapolis, IN (US); Jon Gordon SELBO, Noblesville, IN (US); Rajni Miglani BHARDWAJ, Libertyville, IN (US)

(21) Appl. No.: 18/064,346

(22) Filed: Dec. 12, 2022

Related U.S. Application Data
(60) Provisional application No. 63/422,542, filed on Nov. 4, 2022.
(60) Provisional application No. 63/288,777, filed on Dec. 13, 2021.

(51) Int. Cl. C07D 519/00

(52) U.S. Cl. CPC C07D 519/00; C07B 2200/13

(57) ABSTRACT

Provided herein are methods of preparing crystalline, selpercatinib Form A, which contains little to none of the thermodynamically more stable, crystalline selpercatinib Form B. Selpercatinib is useful in the treatment and prevention of diseases which can be treated with a RET kinase inhibitor, including RET-associated diseases and disorders.

### BACKGROUND

Selpercatinib (LOXO-292 or RETEVMO™) is a RET inhibitor approved in the United States for use in the treatment of patients with metastatic RET fusion-positive NSCLC, RET-mutant medullary thyroid cancer, and RET fusion-positive thyroid cancer. Selpercatinib, or 6-(2-hydroxy-2-methylpropoxy)-4-(6-(6-((6-methoxypyridin-3-yl)methyl)-3,6-diazabicyclo[3.1.1]heptan-3-yl)pyridin-3-yl)pyrazolo[1,5-a]pyridine-3-carbonitrile, has the following chemical structure:

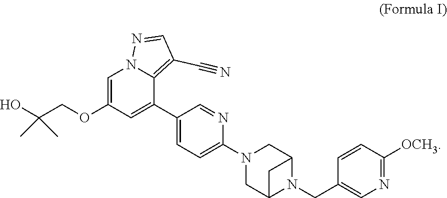 [SMILES: COc1ccc(CN2C3CC2CN(c2ccc(-c4cc(OCC(C)(C)O)cn5ncc(C#N)c45)cn2)C3)cn1]

While several crystal forms of selpercatinib are known and have been disclosed (see, e.g., U.S. Pat. No. 10,584,124) the various crystalline polymorphic forms, when isolated, can include amounts of one or more other crystalline form(s), as a polymorphic impurity. For example, “Form A,” is a crystal form disclosed in U.S. Pat. No. 10,584,124 that typically contains at least some of the thermodynamically more stable, crystalline “Form B.” The Form A material disclosed in the U.S. Pat. No. 10,584,124 patent contained some Form B material. WO 2021/211380, discloses methods for selectively forming selpercatinib Form B. Disclosed herein are methods of selectively forming selpercatinib Form A, which contains little, if any, Form B.

### SUMMARY

Disclosed herein are methods of making selpercatinib in its kinetically stable crystalline form, “Form A”. In embodiments of these methods, the disclosure relates to a method of converting selpercatinib in a solubilized and/or solvated form, to selpercatinib Form A. In other embodiments of these methods, the disclosure relates to a method of converting selpercatinib as a mixture of polymorphic forms, to selpercatinib Form A. In yet other embodiments, the method comprises converting a mixture comprising selpercatinib Form B to Form A.

These crystal forms can be incorporated into formulations, such as tablets, capsules, and suspensions, which can benefit patients. It is also advantageous to be able to provide selpercatinib selected as one of its crystalline forms (e.g., kinetically stable Form A), which can be mixed with one or more other crystalline forms and/or provided as a single crystalline form (i.e., as a pure or substantially pure crystalline form).

As described in more detail below, the compound of Formula I (selpercatinib) can be provided as polymorphic forms (Form A and Form B) and, surprisingly, that certain processes and methods are effective to provide selpercatinib in its kinetically stable polymorph Form A. As described below and demonstrated by the illustrative working examples, the processes and methods for generating and preparing selpercatinib in a specific polymorph form may comprise converting (i.e., reacting, contacting, and/or treating) the compound of Formula I provided as one or more polymorph forms, under crystallization conditions that are effective to generate or convert the other polymorphs (i.e., Form B) or amorphous selpercatinib to Form A. In other aspects, the processes and methods for generating selpercatinib Form A may comprise a synthetic route comprising reacting one or more intermediate or precursor compounds under conditions that are effective to generate selpercatinib Form A (i.e., direct synthetic routes).

In some embodiments of these aspects, Form A as prepared by methods in accordance with the disclosure may be converted to selpercatinib Form B using one or more of the methods as described herein.

Form B is characterized by at least one of (a) an x-ray powder diffraction (XRPD) pattern comprising a peak at 21.1° and one or more peaks at 7.5, 10.9, 12.0°, 17.1°, 17.7°, and 19.8°±0.2° 2θ as measured using an x-ray wavelength of 1.5418 Å, or (b) a ¹³C solid state NMR spectrum which comprises peaks referenced to the high field resonance of adamantane (δ=29.5 ppm) at: 28.0, 48.0, 80.4, 106.8, 130.2, and 134.9 ppm (±0.2 ppm, respectively). Typically, these signature spectra are unique to crystalline Form B.

Similarly, Form A can be identified on the basis of XRPD peaks at 4.9, 9.7, and 15.5°, ±0.2° 2θ that are not observable with Form B, and/or (b) a NMR spectrum comprising a peak (referenced to adamantane (δ=29.5 ppm)) at 30.9 ppm that is not observable with Form B.

Disclosed herein is a method of converting selpercatinib to selpercatinib Form A. Preferably, the selpercatinib contains at least about 92 wt % Form A. More Preferably, the selpercatinib contains at least about 94 wt % to about 98 wt % Form A. The selpercatinib may be amorphous, Form B (the thermodynamically more stable polymorph), a selpercatinib solvate, or a mixture of two or more thereof.

Also disclosed herein is a method for converting selpercatinib to selpercatinib Form A, the method comprising:
a. dissolving selpercatinib in a solvent comprising DMSO and thereby forming a selpercatinib DMSO solution;
b. adding water to the selpercatinib DMSO solution to form a slurry; and
c. isolating the crystallized selpercatinib Form A from the slurry, wherein the Form A has XRPD peaks at about 4.9, 9.7, and 15.5°2θ.

Further disclosed is a method for converting selpercatinib to selpercatinib Form A, the method comprising:
a. dissolving the selpercatinib in a solvent comprising dichloromethane to form a solution;
b. adding heptane to the solution and under conditions effective to form a slurry;
c. isolating the selpercatinib Form A from the slurry, wherein the Form A has XRPD peaks at about 4.9, 9.7, and 15.5°2θ.

It was surprisingly discovered that using the methods described herein to prepare selpercatinib Form A, but using the wrong washing and drying protocol afforded Form A that contained up to about 20 wt % of Form B. Thus, disclosed herein is a method for washing and drying selpercatinib Form A that minimizes or prevents the formation of Form B.

### BRIEF DESCRIPTION OF THE FIGURES

FIG. 1 is an overlay of Form A and Form B XRPD data, up to about 26° two theta (2θ).

FIG. 2 Is a representative HPLC chromatogram used for crystallization development with assignments for the impurities of interest.

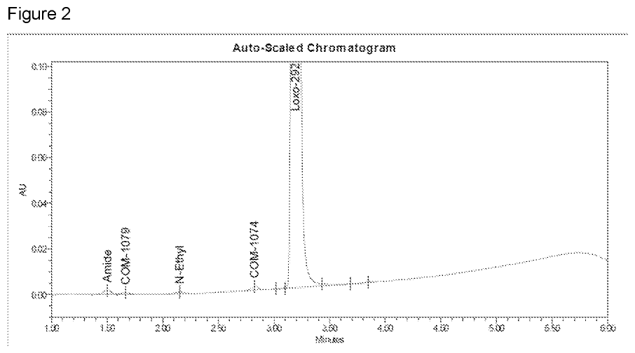

FIG. 3 contains ¹³C solid state NMR data for Form A, Form B, and an overlay of about 25 to 60 ppm that compares Form A to Form B.

### DETAILED DESCRIPTION

Definitions

Unless defined otherwise, all technical and scientific terms used herein have the meaning commonly understood by a person skilled in the art to which this invention belongs. As used herein, the following terms have the meanings ascribed to them below, unless specified otherwise.

The term “polymorph,” as used herein, refers to crystals of the same compound having different physical properties as a result of the order of the molecules in the crystal lattice. Different polymorphs of a single compound (i.e. a compound of Formula I) have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bio-availability), solubility, melting point, chemical stability, physical stability, powder flowability, water sorption, compaction, and particle morphology. Differences in stability can result from changes in chemical reactivity (e.g., differential oxidation, such that a dosage form discolors more rapidly when comprised of one polymorph than when comprised of another polymorph) or mechanical changes (e.g., crystal changes on storage as a kinetically favored polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph”, as used herein, does not include amorphous forms of the compound. In some particular embodiments, the polymorph of the compound of Formula I (i.e., one or both of selpercatinib Form A and/or selpercatinib Form B) comprises the characteristics as described herein.

As used herein, “amorphous” refers to form of a compound which lacks crystalline order. For example, “amorphous” refers to a compound (e.g., a solid form of the compound) without a regularly repeating arrangement of molecules or external face planes and is typically characterized by the lack of sharp diffracting peaks in its powder x-ray diffraction pattern.

The term “anhydrous,” as used herein, refers to a crystal form of the compound of Formula (I) that does not contain stoichiometric amounts of water associated with the crystal lattice. Typically, anhydrous Form A and anhydrous Form B have 1% or less by weight water. For example, 0.5% or less, 0.25% or less, or 0.1% or less by weight water.

The term “solvate” as used herein refers to a crystalline form of the compound of Formula (I), where the crystal lattice includes one or more solvents.

The terms “hydrate” or “hydrated polymorph form” refer to a crystalline form of the compound of Formula (I), such as a polymorph form of the compound, where the crystal lattice includes water. Unless specified otherwise, the term “hydrate” as used herein refers to a “stoichiometric hydrate.” A stoichiometric hydrate contains the water molecules as an integral part of the crystal lattice. In comparison, a non-stoichiometric hydrate comprises water, but changes in the water content does not cause significant changes to the crystal structure. During drying of non-stoichiometric hydrates, a considerable proportion of water can be removed without significantly disturbing the crystal network, and the crystals can subsequently rehydrate to give the initial non-stoichiometric hydrated crystalline form. Unlike stoichiometric hydrates, the dehydration and rehydration of non-stoichiometric hydrates is not accompanied by a phase transition, and thus all hydration states of a non-stoichiometric hydrate represent the same crystal form.

“Purity,” when used in reference to a composition including a polymorph of the compound of Formula (I), refers to the percentage of one specific polymorph form relative to another polymorph form or an amorphous form of the compound of Formula (I) in the referenced composition. For example, a composition comprising polymorph Form A having a purity of 90% would comprise 90 weight parts Form A and 10 weight parts of other polymorph and/or amorphous forms of the compound of Formula (I).

As used herein, a compound or composition is “substantially free of” one or more other components if the compound or composition contains no significant amount of such other components. For example, the composition can contain less than 5%, 4%, 3%, 2%, or 1% by weight of other components. Such components can include starting materials, residual solvents, or any other impurities that can result from the preparation of and/or isolation of the compounds and compositions provided herein. In some embodiments, a polymorph form provided herein is substantially free of other polymorph forms. In some embodiments, a particular polymorph of the compound of Formula (I) is “substantially free” of other polymorphs if the particular polymorph constitutes at least about 95% by weight of the compound of Formula (I) present. In some embodiments, a particular polymorph of the compound of Formula (I) is “substantially free” of other polymorphs if the particular polymorph constitutes at least about 97%, about 98%, about 99%, or about 99.5% by weight of the compound of Formula (I) present. In certain embodiments, a particular polymorph of the compound of Formula (I) is “substantially free” of water if the amount of water constitutes no more than about 2%, about 1%, or about 0.5% by weight of the polymorph.

As used herein, “substantially pure,” when used in reference to a polymorph form of the compound of Formula (I), means a sample of a polymorph form of the compound having a purity greater than 90%, including greater than 90%, 91%, 92%, 93%, 94%, 95%, 96%, 97%, 98%, and 99%, and also including equal to about 100% of the compound, based on the weight of the compound. The remaining material comprises other form(s) of the compound, and/or reaction impurities and/or processing impurities arising from its preparation. For example, a polymorph form of the compound of Formula (I) may be deemed substantially pure in that it has a purity greater than 90% of a polymorph form of the compound of Formula (I), as measured by means that are at this time known and generally accepted in the art, where the remaining less than 10% of material comprises other form(s) of the compound of Formula (I) and/or reaction impurities and/or processing impurities. The presence of reaction impurities and/or processing impurities may be determined by analytical techniques known in the art, such as, for example, chromatography, nuclear magnetic resonance spectroscopy, mass spectrometry, or infrared spectroscopy.

To provide a more concise description, some of the quantitative expressions herein are recited as a range from about amount X to about amount Y. It is understood that when a range is recited, the range is not limited to the recited upper and lower bounds, but rather includes the full range from about amount X through about amount Y, or any range therein.

“Room temperature” or “RT” refers to the ambient temperature of a typical laboratory, which is typically around 20-25° C.

As used herein, the term “excipient” refers to any substance needed to formulate the composition to a desired form. For example, suitable excipients include but are not limited to, diluents or fillers, binders or granulating agents or adhesives, disintegrants, lubricants, antiadherants, glidants, dispersing or wetting agents, dissolution retardants or enhancers, adsorbents, buffers, chelating agents, preservatives, colors, flavors and sweeteners.

The term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is well known in the art. Except insofar as any conventional media or agent is incompatible with the active ingredient, its use in the therapeutic formulations is contemplated. Supplementary active ingredients can also be incorporated into the formulations. In addition, various excipients, such as are commonly used in the art, can be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

As used herein, the singular forms “a,” “an,” and “the” include plural referents unless the context clearly dictates otherwise.

As used herein, ranges and amounts can be expressed as “about” a particular value or range. About also includes the exact amount. Hence, “about 5 grams” means “about 5 grams” and also “5 grams.” It also is understood that ranges expressed herein include whole numbers within the ranges and fractions thereof. For example, a range of between 5 grams and 20 grams includes whole number values such as 5, 6, 7, 8, 9, 10, 11, 12, 13, 14, 15, 16, 17, 18, 19 and 20 grams, and fractions within the range including, but not limited to, 5.25, 6.5, 8.75 and 11.95 grams. The term “about” preceding a value for DSC, TGA, or TG which are reported as degrees Celsius, have an allowable variability of +/−5° C.

As used herein, “optional” or “optionally” means that the subsequently described event or circumstance does or does not occur and that the description includes instances where said event or circumstance occurs and instances where it does not. For example, a reaction mixture that “optionally includes a catalyst” means that the reaction mixture contains a catalyst, or it does not contain a catalyst.

As used herein, the term “dilute,” when used with regard to an acid solution, refers to a solution having an acid concentration of less than about 0.1 N.

The terms “hydrogen” and “H” are used interchangeably herein.

A salt can form from a compound in any manner familiar to the skilled artisan. Accordingly, the recitation “to form a compound or salt thereof” includes embodiments where a compound is formed and the salt is subsequently formed from the compound in a manner familiar to the skilled artisan.

Herein, a patient is one in whom a RET fusion or RET mutation has been determined. As such, the term “determining a RET fusion or RET mutation” means determining if a RET fusion or RET mutation is present. Methods for determining the if a RET fusion or RET mutation is present are known to those of ordinary skill in the art, e.g., see Wang, Yucong et al., Medicine 2019; 98(3): e14120. In embodiments, the term “patient” refers to a human.

A “pharmaceutically acceptable carrier, diluent, or excipient” is a medium generally accepted in the art for the delivery of biologically active agents to mammals, e.g., humans.

The terms “treatment,” “treat,” “treating,” and the like, are meant to include slowing, stopping, or reversing the progression of a disorder. These terms also include alleviating, ameliorating, attenuating, eliminating, or reducing one or more symptoms of a disorder or condition, even if the disorder or condition is not actually eliminated and even if progression of the disorder or condition is not itself slowed, stopped, or reversed.

“Effective amount” means the amount of the crystalline form of selpercatinib that will elicit the biological or medical response of or desired therapeutic effect on a patient by a treating clinician. In one example, the crystalline form of selpercatinib inhibits native RET signaling in an in vitro or ex vivo RET enzyme assay. In another example, the crystalline form of selpercatinib inhibits native RET signaling in mouse whole blood from animals treated with different doses of the compound.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

Selpercatinib, either as Form B or Form A, or mixtures thereof, is preferably formulated as pharmaceutical compositions administered by any route which makes the compound bioavailable, including oral, intravenous, and transdermal routes. Most preferably, such compositions are for oral administration. Such pharmaceutical compositions and processes for preparing same are well known in the art. (See, e.g., Remington: The Science and Practice of Pharmacy (D.B. Troy, Editor, 21st Edition, Lippincott, Williams & Wilkins, 2006).

As used herein, “granulate composition” refers to a composition in granular form which, in the pharmaceutical manufacturing process, is a predecessor composition to a pharmaceutical composition.

As used herein, “manufacturing container” refers to a container that is employed in the manufacture of a pharmaceutical, but not in the medicinal chemistry laboratory. Examples of manufacturing containers include, but are not limited to, a hopper collector, a bed, a dryer bed, a granulator bed, a dryer tray, a granulator bucket, and a mixing bowl.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

All combinations of the embodiments pertaining to the aspects described herein are specifically embraced by the present disclosure just as if each and every combination was individually explicitly recited, to the extent that such combinations embrace possible aspects. In addition, all sub-combinations of the embodiments contained within the aspects described herein, as well as all sub-combinations of the embodiments contained within all other aspects described herein, are also specifically embraced by the present invention just as if each and every sub-combination of all embodiments are explicitly recited herein.

### Methods Providing Crystalline Forms of Selpercatinib

Some non-limiting methods of the disclosure are described below. In some aspects, the disclosure provides methods and processes effective in converting Form A to Form B. Yet other aspects of the disclosure provide methods and processes effective for preparing Form A and/or converting other forms of selpercatinib (e.g., Form B) to Form A.

Form A has unique XRPD peaks at about 4.9, 9.7, and 15.5° 2θ, while Form B has unique XRPD peaks at about 7.5, 10.9, and 12.0° 2θ. The 2θ values and/or peak intensities of other peaks also differ between the two forms, as may be seen in Table 1 below. To be clear, all XRPD peaks disclosed herein are ±0.2°2θ, unless expressly identified otherwise.

TABLE 1

| | |
|---|---|
| Peak position | Relative intensity |

FORM A

| | |
|---|---|
| 18.8 | 24.3% |
| 20.2 | 4.0% |
| 21.0 | 5.7% |
| 21.9 | 6.4% |
| 22.6 | 8.1% |
| 23.6 | 9.1% |
| 25.1 | 7.7% |
| 25.5 | 14.4% |
| 26.0 | 8.9% |
| 26.4 | 6.3% |
| 27.2 | 4.6% |
| 28.2 | 5.6% |
| 28.8 | 3.1% |
| 29.3 | 1.6% |
| 31.5 | 1.5% |
| 32.2 | 1.4% |
| 33.2 | 1.0% |
| 33.7 | 1.4% |

FORM B

| | |
|---|---|
| 18.6 | 1.8% |
| 19.6 | 13.5% |
| 19.8 | 18.8% |
| 20.1 | 6.4% |
| 21.1 | 100.0% |
| 22.5 | 7.6% |
| 24.3 | 3.1% |
| 24.6 | 5.8% |
| 25.0 | 6.1% |
| 26.5 | 2.0% |
| 26.7 | 3.1% |
| 27.7 | 2.0% |
| 28.0 | 2.1% |
| 28.4 | 3.2% |
| 28.9 | 4.1% |
| 29.2 | 7.5% |
| 30.0 | 7.7% |
| 30.3 | 3.3% |
| 32.6 | 1.4% |
| 33.2 | 2.7% |
| 34.1 | 1.3% |
| 34.3 | 1.3% |
| 35.3 | 1.0% |

The XRPD data was obtained on a Bruker D4 Endeavor X-ray powder diffractometer, equipped with a CuKa source (λ=1.54180 Å) and a Vantec detector, operating at 35 kV and 50 mA. The sample is scanned between 4 and 40 2θ°, with a step size of 0.008 2θ° and a scan rate of 0.5 seconds/step, and using 1.0 mm divergence, 6.6 mm fixed anti-scatter, and 11.3 mm detector slits. The dry powder is packed on a quartz sample holder and a smooth surface is obtained using a glass slide. The crystal form diffraction patterns are collected at ambient temperature and relative humidity. Crystal peak positions are determined in MDI-Jade after whole pattern shifting based on an internal NIST 675 standard with peaks at 8.853 and 26.774 2θ°. It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 20° is presumed to take into account these potential variations without hindering the unequivocal identification of the indicated crystal form. Confirmation of a crystal form may be made based on any unique combination of distinguishing peaks.

DSC-TGA analyses of an anhydrous, crystalline Form A demonstrated a melting onset of about 207° C. and exhibited two endotherms, where the first endotherm corresponds to the melt of Form A followed by the exothermic recrystallization of Form B and then the melt of Form B. DSC-TGA analyses of an anhydrous, crystalline Form B demonstrated a single endotherm with a melting onset of about 213° C.

While Forms A and B are anhydrous polymorphs, Form A is slightly more hygroscopic than Form B and, as discussed herein, is thermodynamically less stable than Form B. Furthermore, and as discussed herein, some embodiments provide selpercatinib in the form of a solvate, which may be isolated. In some embodiments, removal of the solvent molecule(s) from selpercatinib in solvated form can provide for selpercatinib Form A.

Forms A and B have similar solubilities. Both exhibit poor 25° C. solubility in many organic solvents, including methyl ethyl ketone (MEK), acetone, and many alcohol based solvents, while having moderate solubility (3-30 mg/ml) in dichloromethane (DCM), dimethylsulfoxide (DMSO) and THF. Form B has almost no solubility in anisole.

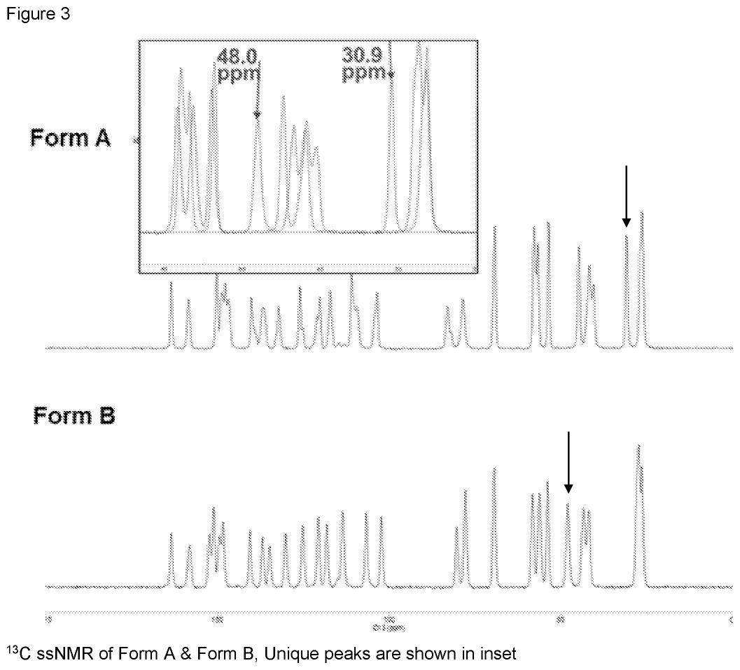

The ¹³C solid state NMR spectra of Forms A and B appear in FIG. 3. FIG. 3 also contains an overlay of a portion of the spectra, which shows Form A has a peak at 30.9 ppm that is not observable in Form B, while Form B has a peak at about 48.0 ppm that is not observable in Form A. Both spectra were referenced to the high field resonance of adamantane (δ=29.5 Ppm).

¹³C Cross polarization/magic angle spinning NMR (solid-state NMR or ssNMR) spectra referenced above were obtained using a Bruker Avance III HD 400 MHz wide-bore NMR spectrometer operating at a carbon frequency of 100.62 MHz and proton frequency of 400.13 MHz, and equipped with a Bruker 4 mm double resonance probe. TOSS sideband suppression was used along with cross polarization employing SPINAL64 decoupling and a RAMP 100 shaped H-nucleus CP pulse. Acquisition parameters were as follows: 4.0 μs proton pulse, 1.5 ms contact time, 5 kHz MAS frequency, 30.2 kHz spectral width, and 34 ms acquisition time. A 3 second recycle delay is used and the number of scans is 2655. Chemical shifts are referenced to adamantane (δ=29.5 ppm) in a separate experiment. Representative ¹³C ssNMR resonances for Form B include peaks at about: 26.44, 27.37, 28.00, 41.98, 43.43, 43.91, 48.04, 53.92, 56.31, 58.32, 69.48, 77.90, 80.38, 102.32, 106.77, 113.58, 115.24, 118.23, 120.76, 125.23, 130.23, 134.86, 136.93, 140.59, 148.42, 149.50, 151.20, 152.45, 158.22, and 163.52 ppm. As illustrated, Form A has a peak at about 30.9 ppm that is not observable in Form B.

The above data establishes that Forms B and A: 1) have some different properties, 2) can readily be identified and distinguished from each other based on such properties, 3) Form A can be prepared by the methods describe herein, and as discussed in aspects and embodiments below, 5) Form A can be prepared and/or converted from selpercatinib in other forms, including from solvates and/or Form B.

Given the similar solubility between selpercatinib Form A and Form B, a number of suitable solvents can be used in accordance with the aspects and embodiments of the disclosure. In some embodiments, solvents and/or process conditions can be used and adjusted so that the resulting crystalline form can be predominantly Form A (e.g., pure or substantially pure Form A).

As noted above, selpercatinib can form solvates; and it can also form metastable solid forms, both of which are generally not stable on drying. Observed solvates include the acetone solvate, chloroform solvate, 1,4-dioxane solvate, methyl ethyl ketone (MEK) solvate, dichloromethane (DCM) solvate, 2-butanol solvate, 1-butanol solvate, ethanol solvate, dimethylsulfoxide (DMSO)-water solvate, DMSO solvate, isopropyl alcohol (IPA) solvate and the tetrahydrofuran (THF) solvate. The solvates and metastable forms usually revert to Form A during isolation and/or drying, although films or amorphous material occasionally form. The chloroform and 1,4-dioxane solvates were stable upon isolation/drying. Thus, one strategy for preparing selpercatinib Form A is to convert amorphous selpercatinib and/or selpercatinib Form B into a solvate and then desolvating the solvate, to afford Form A.

In embodiments of the methods described herein for preparing Form A, the selpercatinib may comprise an amount of Form B and/or an amount of Form A.

In an aspect, selpercatinib Form A is described herein. This crystalline form of selpercatinib could be used to treat disorders associated with abnormal RET activity, e.g., IBS or cancer, especially cancer stemming from overactive RET signaling (i.e., RET-associated cancers). More specifically, this crystalline form of selpercatinib could be used to treat RET-associated cancers such as lung cancer (e.g., small cell lung carcinoma or non-small cell lung carcinoma), thyroid cancer (e.g., papillary thyroid cancer, medullary thyroid cancer, differentiated thyroid cancer, recurrent thyroid cancer, or refractory differentiated thyroid cancer), thyroid ademona, endocrine gland neoplasms, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, mammary cancer, mammary carcinoma, mammary neoplasm, colorectal cancer (e.g., metastatic colorectal cancer), papillary renal cell carcinoma, ganglioneuromatosis of the gastroenteric mucosa, inflammatory myofibroblastic tumor, or cervical cancer.

Form A may be used in a method for treating cancer, comprising administering an effective amount of Form A to a patient in need thereof. The types of cancers that may be treated using the methods described herein include hematological cancer or solid tumor cancer. Examples of the types of cancer that may be treated using Form B include lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer. Specifically, the types of cancer can be lung cancer or thyroid cancer. More specifically, the cancer can be non-small cell lung carcinoma or medullary thyroid cancer.

Also described herein is Form A, for use in therapy.

Form A may be used in the manufacture of a medicament for the treatment of RET-associated diseases or disorders such as IBS or cancer. Cancers that can be treated using such a medicament are described herein above. Use of Form A in the manufacture of a medicament may also include a step of performing an in vitro assay using a biological sample from a patient, determining the presence of a dysregulation of a RET gene, a RET kinase, or expression or activity or level of any of the same, and administering a therapeutically effective amount of Form A, to the patient if a dysregulation of a RET gene, a RET kinase, or expression or activity or level of any of the same is present. In these uses, the biological sample can be a tumor sample and the tumor sample can be analyzed using methods known to those of skill in the art such as genomic/DNA sequencing. Additionally, in these uses the sample can be obtained from the patient prior to the first administration of Form A. In these uses of Form B, as described herein in a therapy can be based upon a patient being selected for treatment by having at least one dysregulation of a RET gene, a RET kinase, or expression or activity or level of any of the same. Also, in these uses Form A may be administered to the patient at a dose of about 1 mg/kg to 200 mg/kg (effective dosage sub-ranges are noted herein above).

### Selpercatinib Form A—Compositions, Compounds, and Processes

As described herein, selpercatinib Form A can contain an amount of the thermodynamically stable polymorph selpercatinib Form B. While both polymorph forms are crystalline, high-melting, anhydrous, stable, and do not readily inter-convert under typical storage or preparative conditions, the polymorphs have different properties and characteristics, which allows Form A to be distinguished from Form B. Given the differences in the favored thermodynamic stability of selpercatinib Form A and Form B, there is a need to understand how to convert and generate from either form to the other (e.g., Form B to Form A, as described below).

### Crystallization Methods Providing Form A

In an aspect, the disclosure provides methods of preparing selpercatinib Form A, including methods that convert amorphous selpercatinib and/or selpercatinib in other polymorphic forms, including mixtures of forms (e.g., comprising selpercatinib Form B) to selpercatinib Form A. While selpercatinib Form A can be prepared or converted from other selpercatinib forms using a variety of different methods, disclosed herein are crystallization-based methods that prepare or convert selpercatinib in other crystalline forms (e.g., comprising selpercatinib Form B) to selpercatinib Form A.

Suitable methods for preparing Form A include but are not limited to cooling crystallization, evaporation crystallization, vapor diffusion, crystallizations using one or more antisolvents (including forward or reverse antisolvent addition, co-additions or continuous crystallization), and slurry crystallization. Suitable methods also include using washing and drying methods to help to minimize or prevent the formation of Form B material. These methods are discussed herein.

In one aspect, disclosed herein is a method of converting a mixture of selpercatinib that comprises Form B, to selpercatinib Form A.

In one aspect, disclosed herein is a method of converting amorphous selpercatinib to selpercatinib Form A.

In another aspect, disclosed herein is a method of converting selpercatinib in another form or a mixture of other forms (e.g., comprising Form B) to selpercatinib Form A, the method comprising: combining selpercatinib that includes Form B with DMSO and water to generate a slurry and isolating selpercatinib Form A from the slurry.

In yet another aspect, disclosed herein is a method for converting selpercatinib (e.g., Form B) to selpercatinib Form A, the method comprising:
a. dissolving the selpercatinib in a solvent comprising DMSO to form a solution;
b. adding water to the solution and thereby forming a slurry comprising selpercatinib Form A;
c. isolating the selpercatinib Form A.

In an embodiment, about 1 gram of selpercatinib is dissolved in about 10-15 mL of DMSO. In another embodiment, forming the solution of step a comprises heating the selpercatinib and the solvent comprising DMSO to about 50° C. to about 70° C. In one embodiment, after heating the solution to about 50° C. to about 70° C., the solution is cooled to a temperature less than about 70° C. and greater than about 20° C. In an embodiment, the solution is cooled to about 40° C. In another embodiment, step b comprises adding about 0.1 to about 1 mL/g of water to the solution or step b comprises adding about no more than about 0.2 mL/g of water to the solution. In some embodiments, step b further comprises adding about 1 to about 15 wt % of Form A seed crystals or about 1 wt % of Form A seed crystals. In an embodiment, the slurry is cooled to about 0° C. In one embodiment, the adding of water to form the slurry of step b comprises addition of two separate volumes of water, to a total DMSO:water ratio of no more than 80:20. In an embodiment, step c comprises filtration. The isolated selpercatinib Form A from step c is washed with a solvent comprising MTBE and/or water.

In yet another aspect, disclosed herein is a method for converting selpercatinib (e.g., Form B) to selpercatinib Form A, the method comprising:
a. Dissolving the selpercatinib in a solvent comprising DMSO to form a solution
b. Adding the selpercatinib/DMSO solution to a solution of water or

DMSO/water and thereby forming a slurry comprising selpercatinib Form A:
c. Isolating the selpercatinib Form A.

In an embodiment, about 1 gram of selpercatinib is dissolved in about 10-15 mL of DMSO. In another embodiment, forming the solution of step a comprises heating the selpercatinib and the solvent comprising DMSO to about 50° C. to about 70° C. In one embodiment, after heating the solution to about 50° C. to about 70° C., the solution is cooled to a temperature less than about 70° C. and greater than about 20° C. In an embodiment, the solution is cooled to about 40° C. In another embodiment, step b comprises adding the solution of step a to at least one volume of water or DMSO/water. In some embodiments, step b further comprises adding about 1 to about 15 wt % of Form A seed crystals or about 1 wt % of Form A seed crystals. In an embodiment, the slurry is cooled to about 0° C. In one embodiment, at the end of step b, the DMSO:water ratio is about 80:20. In an embodiment, step c comprises filtration. The isolated selpercatinib Form A from step c is washed with a solvent comprising MTBE and/or water.

In another aspect, disclosed herein is a method for converting selpercatinib (e.g., Form B) to selpercatinib Form A, the method comprising:
a. Dissolving the selpercatinib in a solvent comprising DMSO to form a solution (Feed 1)
b. Preparing a water or DMSO/water solution (Feed 2)
c. Adding the selpercatinib/DMSO solution (Feed1) simultaneously with Feed 2 to a solution of water or DMSO/water and thereby forming a slurry comprising selpercatinib Form A:
d. Isolating the selpercatinib Form A.

In another aspect, disclosed herein is a method for converting selpercatinib (e.g., selpercatinib that includes Form B) to Form A, the method comprising: combining selpercatinib and dichloromethane to form a solution, adding heptane to the solution under conditions to form a slurry, optionally stirring the slurry under conditions effective to form selpercatinib Form A, and isolating selpercatinib Form A. In an embodiment, about 1 gram of selpercatinib is dissolved in about 25-35 mL/g of dichloromethane. In one embodiment, forming the solution of step a comprises heating the selpercatinib and the solvent comprising dichloromethane to about 30° C. to about 40° C. In a further embodiment, step b comprises adding a first batch of heptane and a second batch of heptane. In some embodiments, the adding of heptane comprises adding a first volume of heptane in an amount of about 8-12 mL/g selpercatinib, and a second volume of heptane in an amount of about 8-12 mL/g. In an embodiment, the solution of step b is cooled to a temperature of less than about 30° C. and greater than about 20° C., or more preferably, the solution is cooled to a temperature of about 25° C. Step b may comprise stirring for at least about 8 h.

A variety of different solvents can be used to prepare Form A and/or convert other forms of selpercatinib (e.g., Form B) to Form A. In some aspects and embodiments, the solvent may combine with the selpercatinib to generate a solvate. Solvents that can be used to prepare Form A and/or convert other selpercatinib forms (e.g., Form B) to Form A include, but are not limited to C₁-C₆ alcohols (such as methanol or ethanol), water, acetonitrile (ACN), methyl tert-butyl ether (MTBE), dicholoromethane (DCM), heptane, n-butyl acetate (n-BuOAC), 81% ACN-MeOH (81 mL ACN combined with 19 mL MeOH), wet ethyl acetate, cyclopentyl methyl ether (CPME), 1,2-dimethoxyethane, ethyl acetate, ethyl formate, methyl isobutyl ketone (MIBK), nitromethane, n-propyl acetate (NPA), 1-pentanol, toluene, 1:1 MeOH:water, 1:1 EtOH:water, ACN:water, DCM/heptane mixtures, DMSO/heptane mixtures, or DMSO/water mixtures. While using C₁-C₆ alcohols, such as methanol and/or ethanol will convert Form B to Form A, they can also lead to the Formation of Form B. As detailed below, washing the Form A with a C₁-C₆ alcohol may lead to the formation of Form B. If a C₁-C6 alcohol is used to wash the Form A, using cold C₁-C₆ is preferred.

It was surprisingly and unexpectedly found that Form B material may form during the washing and drying of the Form A material. To reduce, if not prevent the formation of Form B material, the following washing and drying protocol was developed. After forming a solvate, wash the solvate with a solvent, such as heptane or MTBE, and then dry the resulting cake at about 40 to about 60° C. In an embodiment, the cake is dried under vacuum. When dried under vacuum, lower drying temperatures may be used. For example, when drying a cake of Form A under vacuum, a temperature of about 40 to 45° C. may be used. The heptane and MTBE may be used individually or sequentially. Excess temperatures and/or excess drying times can allow the kinetic product, Form A, to convert to the thermodynamic product, Form B.

The inventors discovered drying the Form A wet cake at 45° C. and at ambient pressured over several days slowly converted the Form A to Form B. Drying under vacuum and/or using MTBE as the final wash reduced the drying time and reduced, if not prevented, the formation of any Form B material. In one preferred embodiment, the Form A material is washed with MTBE or heptane and then dried under vacuum at a temperature of about 40 to about 45° C.

Further, the inventors discovered using water, MeOH and finally MTBE to wash the Form A cake and then drying the resulting cake under vacuum afforded up to about 20 wt % of Form B material. Without wishing to be bound by a theory, it is believed that washing with MeOH accelerates the formation of Form B material.

In some embodiments the methods and processes for preparing Form A can include a non-liming solvent that includes C₁-C4 alcohols, water, DCM, DMSO, MTBE, ACN and mixtures of two or more thereof. In still other embodiments of such methods, the solvent comprises methanol, ethanol, water, DMSO, MTBE, ACN or mixtures of two or more thereof. In yet further embodiments of such methods, the solvent comprises DCM, heptane, DMSO, water, MTBE or mixtures of two or more thereof.

In various aspects, the methods comprise combining selpercatinib, e.g., selpercatinib comprising an amount of Form B with a solvent, and heating the resulting mixture, optionally with stirring or mixing until the selpercatinib comprising Form B dissolves in the solvent. Once a solution is formed, the mixture may be filtered, if any insoluble impurities are to be removed, and cooled, e.g., slightly above or at room temperature (e.g., about 25-40° C., depending on solvents used). Additional solvent(s) may added during or after the cooling.

In some embodiments of these aspects, the solvent comprises DMSO and water is added to the solution during or after the cooling step. Once an amount of water is added to the cooled solution, seed crystals comprising selpercatinib may be added, either in dry form or as a slurry in a minimal volume of liquid, and are incubated for a period of time. After the incubation period, (e.g., about 40° C.) additional water is slowly added. After the addition of the water, the mixture is cooled gradually to a target temperature of about 0° C. Once at the target temperature, the slurry or mixture is incubated for a period of time to promote formation of additional solid product. After the incubation period, the resulting selpercatinib Form A material is isolated, and optionally washed to remove residual water and DMSO content. Examples of washing solvents include, but are not limited to, heptane and MTBE. After washing, the Form A material may be dried at a temperature of about 40 to about 60° C. and at a pressure that is below atmospheric pressure up to and including atmospheric pressure. In one preferred embodiment, the pressure is below atmospheric pressure.

In some alternative embodiments of these aspects, the solvent comprises a solvent that forms a solvate of selpercatinib Form A. In some embodiments, the solvent comprises dichloromethane and heptane is added to the solution, and upon addition of the heptane, the mixture is cooled (e.g., to about room temperature/25° C.). After the initial cooling, additional heptane is added and the resulting mixture is stirred for a period of time (e.g., at least 8 h.) at room temperature/25° C. After the stirring, the resulting selpercatinib Form A material is isolated and optionally washed to remove residual dichloromethane.

Solvents

A variety of different solvents may be used in the processes provided by these aspects and embodiments of the disclosure. The solvent, or solvent system, may solubilize and/or form solvated forms of selpercatinib to afford the desired Form A. Examples of suitable solvents include, but are not limited to DMSO, C₁-C₆ alcohols, ACN, MTBE, dichloromethane, water or combinations of two or more thereof. Non-limiting examples of C₁-C₆ alcohols include methanol, ethanol, propanol, and isopropanol. In some embodiments, DMSO is a solvent. In some embodiments, the solvent comprises an amount of DMSO and water, e.g., from about 2% or about 4% to about 20% water (by volume).

The amount of solvent used depends on the solvent that is used. Typically, 1 g of selpercatinib, e.g., comprising an amount of Form B is dissolved in about 8-20 mL, or about 10-15 mL, or about 11-14 mL or about 12-13 mL of solvent used (e.g., about 8, 9, 10, 11, 12, 13, 14, 15, 16, 17, 18, 19, or about 20 volumes of solvent relative to the weight of selpercatinib). In some embodiments, 1 gram of selpercatinib can be dissolved in 8-15 mL/g of DMSO or 1 gram of selpercatinib can be dissolved in about 11-13 mL/g of DMSO or 1 gram of selpercatinib can be dissolved in about 10-15 mL/g of DMSO.

Temperature

Temperature can affect the rate at which the initial selpercatinib (e.g., comprising Form B) is converted to Form A. In some embodiments, the mixture comprising selpercatinib and the solvent is heated in initial steps to a temperature that is at least about 70° C. and up to the boiling point of the solvent. In some embodiments, the mixture is heated to a temperature of about 50-110° C. or about 50° C. to about 70° C. In some embodiments, the mixture may be heated to about 50° C., about 60° C., about 70° C., about 80° C., about 90° C., about 100° C., or about 110° C. After the mixture is heated to the desired temperature and the starting selpercatinib (comprising Form B) material is dissolved, the temperature of the solution is reduced by about 15-40° C. (e.g., prior to the addition of the first tranche of water, discussed below). The temperature may be reduced by about 15° C., about 20° C., about 25° C., about 30° C., or about 35° C. In an embodiment, the solution is cooled to a temperature less than about 70° C. and greater than about 20° C., and in some embodiments to less than 50° C. (e.g., to about 45° C., 44° C., 43° C., 42° C., 41° C., 40° C., 39° C., 38° C., or about 37° C.). In some embodiments, the cooling is performed over a set period of time (i.e., controlled cooling) at a rate of about 5° C./h, 10° C./h, 15° C./h, 20° C./h, 25° C./h, or about 30° C./h.

In some embodiments, the solvent comprises DMSO and the selpercatinib/DMSO mixture is heated to about 60° C. to about 70° C. In a further embodiment, the DMSO is then cooled to about 35° C. to about 45° C., or to about 40° C.

In some alternative embodiments the solvent may not be heated to as high of a temperature as noted above, i.e., the selpercatinib is mixed with solvent, such as dichloromethane, and is allowed to stir at temperatures slightly above ambient temperatures, (e.g., from about 35° C. about 40-50° C.) but which are effective to solubilize selpercatinib. In some embodiments, the temperature is selected to favor the kinetically stable form of selpercatinib (Form A) and to reduce the potential for kinetic turnover. In such embodiments, the temperatures may be selected toward the lower ends of the temperature ranges identified above (e.g., about 40° C.).

First Tranche of Antisolvent

In some embodiments, the methods comprise addition of an antisolvent such as water. In such embodiments, the addition of antisolvent (e.g., heptane or water, depending on the initial solvent used) may comprise multiple additions of separate volumes of antisolvent (e.g., added in tranches). In embodiments comprising addition of water, when the first tranche of water is added to the solution, about 0.1-1.0 mL/g, or about 0.2-0.6 mL/g, or about 0.3 mL/g, or about 0.4 mL/g, or about 0.5 mL/g, or about 0.6 mL/g, of water to Form A is added (mL of water to g of selpercatinib (e.g., Form B)). Stated alternatively, the first addition of water may comprise about 0.1 to about 1.0 volumes of water (i.e., to wt of selpercatinib). In some embodiments, the first tranche of water is added in an amount of about 0.3 mL/g, about 0.4 mL/g, about 0.5 mL/g or about 0.6 mL/g.

The first tranche of water is added over a period of time from about 30 seconds to about 15 minutes or about 1-10 minutes or about 4-6 minutes or about 5 minutes. Longer times may be utilized, if desired. The addition of the first tranche of water is performed under conditions that are effective to avoid any self-seeding of the solution, and typically producing a final solvent-to-water ratio of about 93:7 to about 99:1 (e.g., 99:1, 98:2, 97:3, 96:4, 95:5, 94:6, or 93:7).

In other embodiments that comprise anti solvents other than water (e.g., heptane), the first tranche addition typically comprises a larger volume, typically in an amount of about 30-60% of the total volume of the initial solvent used to form the selpercatinib solution.

Seed Crystals

Form A seed crystals may be added to the mixture when a target temperature is equilibrated in the solution, typically added in amounts of about 0.1-15 wt % or about 1 to about 10 wt % or about 1 to about 5 wt %, or about 1 wt %, 2 wt %, 3 wt %, or about 4 wt % of Form A seed crystals to the initial amount of selpercatinib. In some embodiments, about 0.1 wt %, 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, or about 1.5 wt % of seed crystal is added.

In some embodiments, the temperature at addition of seed crystal is selected to favor the kinetically stable form of selpercatinib (Form A) and to reduce the potential for kinetic turnover. In such embodiments, the temperatures may be selected toward the lower ends of the temperature ranges identified above (e.g., about 40° C.).

The seed crystals can be prepared using the methods known in the art, such as those described in A. Cote, E. Sirota, A. Moment, “The Pursuit of a Robust Approach for Growing Crystals Directly to Target Size” American Pharmaceutical Review—The Review of American Pharmaceutical Business & Technology, 2010, and D. J. Lamberto et. al., “Crystallization Process Development for the Final Step of the Biocatalytic Synthesis of Islatravir: Comprehensive Crystal Engineering for a Low-Dose Drug,” Organic Process Research &

Development 2021 25 (2), 308-317. For example seed crystals may be prepared, obtained, and/or isolated from a source of purified material including, for example, pure selpercatinib Form A, including, e.g., optically or polymorphically pure material. In some embodiments, the seed crystals may obtained or sourced from a prior source of seed crystals. In yet some other embodiments, the seed crystals may be processed, for example, to provide for homogeneous seed crystal material (e.g., jet milling to a desired D₅₀, D₉₀, etc. crystal size). In some embodiments the seed crystals may comprise a D₉₀, of about 1 um to about 10 um (e.g., about 1, 2, 3, 4, 5, 6, 7, 8, 9, or about 10 μm).

Seed Crystal Incubation Time

After the initial heating and cooling of mixture comprising starting selpercatinib (e.g., Form A), and addition of seed crystals, if any, the solution is allowed to incubate for about 30-300 minutes, or about 30-180 minutes, or about 30-120 minutes, or about 30-60 minutes. In some embodiments, the mixture is incubated for no more than about 30 minutes.

Second Tranche of Antisolvent

In some embodiments, after an incubation period of about 30 minutes or more, the mixture is heated to a target incubation temperature of about 35° C. to about 50° C., or about 35° C. to about 45° C., or to about 40° C. Once the target incubation temperature is equilibrated, a second tranche of water is slowly added. The amount of water in the second tranche is from about 0.1-3 mL/g, or about 1.0-2.5 mL/g, or about 1.1 mL/g, about 1.2 mL/g, 1.3 mL/g, about 1.4 mL/g, 1.5 mL/g, about 1.6 mL/g, 1.7 mL/g, about 1.8 mL/g, 1.9 mL/g, about 2.0 mL/g, about 2.1 mL/g, about 2.2 mL/g, 2.3 mL/g, about 2.4 mL/g, 2.5 mL/g, about 2.6 mL/g, 2.7 mL/g, about 2.8 mL/g, 2.9 mL/g, or about 3.0 mL/g of water to amount of initial selpercatinib material is added (mL of water tog of selpercatinib (e.g., Form B)). Stated alternatively, the second addition of water may comprise about 0.1 to about 3.0 volumes of water (i.e., to wt of selpercatinib). In some embodiments, the first tranche of water is added in an amount of about 2.0 mL/g, 2.1 mL/g, 2.2 mL/g, 2.3 mL/g, 2.4 mL/g, 2.5 mL/g or about 2.6 mL/g. In some embodiments, the second tranche of water is added at 2.5 volumes. The resulting amount of water in the resulting solution after the addition of the second tranche of water is complete is about 80:20 (solvent:water, by volume).

The second tranche of water is added over a period of time typically at a slow rate of from about 10 minutes to about 5 h, or about 4 h., about 3 h., about 2 h., about 30-90 minutes or about 45-60 minutes, or about 60 minutes. Longer times may be used, if desired. As noted above, the addition of the second tranche of water is effective to typically produce a final solvent-to-water ratio (by volume) of about 90:10 to about 75:25 (e.g., 90:10, 85:15, 80:20, 75:25).

In some other embodiments, the methods do not comprise addition of seed crystal, and the addition of antisolvent is effective to form the selpercatinib Form A product. In some of these other embodiments, after the addition of the first tranche of antisolvent, the mixture may be cooled to a target temperature (e.g., to ambient temperature) and upon reaching the target temperature a second tranche of antisolvent is added in an amount effective to form selpercatinib Form A (e.g., in a volume about equal to the first tranche of antisolvent). In such embodiments, after the addition of the second tranche of antisolvent, the mixture may be incubated, with stirring for a period of time to provide crystallized selpercatinib Form A.

Cooling

In some embodiments, after the second tranche of water is added, the mixture is cooled over a period of time to a temperature of about 0° C. and forms a slurry. In some embodiments, the mixture is cooled to 0° C., and is maintained at that target temperature for at least about 60 min. (e.g., about 60, 70, 80, 90, 100, 110, or about 120 min).

After the second tranche of water is added, the mixture is cooled at a rate of about 1-30° C./hr, (e.g., at a rate of about 10-30° C./h, e.g., or about 20° C./h) until the desired temperature is reached. In one embodiment, the rate of cooling is about 10° C./hr, about 11° C./hr, about 12° C./hr, about 13° C./hr, about 14° C./hr, about 15° C./hr, about 16° C./hr, about 17° C./hr, about 18° C./hr, about 19° C./hr, or about 20° C./hr.

Isolating Form A

The Form A material may be isolated using any method known in the art. In an embodiment, the separation comprises gravity filtration. In another embodiment, the separation comprises vacuum filtration. In still another embodiment, the separation comprises the use of a centrifugal separation.

Fresh solvents, such as ethanol, methanol, ACN, MTBE, water or combinations of two or more thereof, can be used to wash the Form A material. As previously noted, if ethanol and/or methanol are used to wash the Form A material, then they should be cold, e.g., around 0° C. In some embodiments, DMSO, methanol, ACN, MTBE, water or combinations of two or more thereof, are used to wash the Form A material. In still further embodiments, a solvent comprising DMSO/water (80:20 DMSO:water) is used. In some further embodiments, MTBE may be used to wash any residual solvent (e.g., DMSO/water) to provide the final Form A material. In these embodiments, the fresh solvent may be cooled to a temperature of about 0° C. to less than about 20° C., before it is used to wash the Form A material. In these embodiments, the final wash solvent can be a volatile solvent, such as MTBE, which aids in reducing the solvent hold-up of the cake after filtration and reduces the required drying time. The use of a volatile solvent can also allow for the use of reducedtemperature, which helps to reduce, if not prevent, the formation of Form B material. Excess drying time and/or temperatures can lead to the formation of Form B.

The isolated selpercatinib Form A may be dried using methods known in the art. Typical methods include heating, passing an inert gas over the solid and/or the use of pressures less than atmospheric pressure. In one embodiment, drying under pressure less than atmospheric is preferred.

In embodiments where the solvent comprises DMSO and/or DMSO/water, the isolated selpercatinib Form A may be washed with MTBE until the isolated selpercatinib Form A contains less than 0.5 wt % DMSO (or DMSO/water).

The selpercatinib starting material used in accordance with any of the aspects and embodiments described herein can be purchased from a commercial source, prepared by known synthetic methods, and/or converted from a source of selpercatinib (i.e., amorphous selpercatinib, selpercatinib API, or selpercatinib in another polymorphic form, e.g., one of Form A, Form B, or mixtures thereof).

In aspects relating to Form A, the selpercatinib provided by the disclosure can exhibit greater kinetic stability relative to selpercatinib in its other polymorphic and/or amorphous forms (e.g., Form B).

In any of the aspects and embodiments provided herein, the selpercatinib provided by the disclosure may be prepared as the free amine. Regardless of whether the methods described herein are used to prepare selpercatinib in a particular crystalline form (e.g., selpercatinib Form A), and such form(s) is obtained by direct synthetic method or conversion from selpercatinib (i.e., amorphous selpercatinib or selpercatinib in another polymorphic form) according to aspects and embodiments in accordance with the disclosure, it can be further provided as a pharmaceutically acceptable salt thereof, or pharmaceutical composition thereof. Thus, depending on the particular form, such compounds, salts, and compositions may comprise crystalline selpercatinib that can exhibit greater thermodynamic stability relative to selpercatinib in its other polymorphic and/or amorphous forms, or it can exhibit greater kinetic stability relative to selpercatinib in its other polymorphic and/or amorphous forms. Selpercatinib, in either Form A or Form B retains its activity as a RET inhibitor, and can be evaluated and assessed for activity by any assays known in the art including those assays described in, e.g., PCT Publication No. WO2018/071447 and U.S. Patent Application Publication No. US 20180134702, each of which is incorporated by reference in its entirety. In an embodiment, the selpercatinib Form A is the tosylate or besylate salt. More preferably, when the Form A material is a salt, the salt is the tosylate salt.

Also disclosed herein are pharmaceutical compositions comprising selpercatinib Form A made according to any of the methods disclosed herein. The pharmaceutical compounds may further comprise at least one pharmaceutically acceptable carrier, diluent, or excipient. In some embodiments, the pharmaceutical composition contains less than about 20% by wt. of other crystal forms of selpercatinib or contains less than about 10% by wt. of other crystal forms of selpercatinib or contains less than about 5% by wt. of other crystal forms of selpercatinib. The pharmaceutical composition contains about 40 mg or about 80 mg of selpercatinib Form A. Other pharmaceutical compositions contain about 120 or about 160 mg of selpercatinib Form A. The pharmaceutical formulation may be in a tablet. Alternatively, the pharmaceutical formulation may be in a capsule.

Further, disclosed herein are methods of treating cancer in a patient comprising administering to a patient in need of such treatment an effective amount of selpercatinib Form A made according to any of the methods disclosed herein or a pharmaceutical composition as described herein. In a preferred embodiment, the cancer is a RET associated cancer. RET associated cancers are cancers that respond to inhibition of RET.

In an embodiment, cancers that can be treated using Form A and the compositions described herein are selected from the group consisting of: solid tumor, lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer. In one embodiment, the cancer is medullary thyroid cancer. In another embodiment, the cancer is lung cancer and the lung cancer is small cell lung carcinoma, non-small cell lung cancer, bronchioles lung cell carcinoma, RET fusion lung cancer, or lung adenocarcinoma. In another preferred embodiment, the cancer is solid tumors. In some embodiments, the solid tumors are locally advanced or metastatic solid tumors. In a further embodiment, the solid tumors are locally advanced or metastatic solid tumors with a RET gene fusion that have progressed on or following prior systemic treatment or who have no satisfactory alternative treatment options. In another embodiment, the cancer is locally advanced or metastatic non-small cell lung cancer (NSCLC) with a rearranged during transfection (RET) gene fusion, as detected by an FDA-approved test. In still another embodiment, the cancer is advanced or metastatic thyroid cancer with a RET gene fusion, as detected by an FDA-approved test, who require systemic therapy and who are radioactive iodine-refractory (if radioactive iodine is appropriate).

The Examples that follow are provided merely for purposes of illustrating and describing certain embodiments falling within the scope of the methods described herein, and an encompassed by the claims.

### EXAMPLES

The selpercatinib (6-(2-hydroxy-2-methylpropoxy)-4-(6-(6-((6-methoxypyridin yl)methyl)-5 3,6-diazabicyclo[3.1.1]heptan-3-yl)pyridin-3-yl)pyrazolo[1,5-a]pyridine carbonitrile) used in the crystallization procedures described herein was made using the techniques and methods described in U.S. Pat. No. 10,112,942.

### Example 1

### Gram-Scale Cooling Crystallization Process to Produce Form A

Using a chemical synthesis reactor (Easymax, Mettler Toledo) approximately 5 g of selpercatinib is charged into a reactor along with (11 volumes) DMSO and is heated at 70° C. until the selpercatinib dissolves and the system reaches the 70° C. target temperature. Through heated transfer lines, the solution may be optionally polish filtered prior to transfer to the crystallizer. The reactor and transfer lines are rinsed with (1 volume) DMSO, which is charged to the crystallizer and combined with the selpercatinib solution. The resulting solution is cooled to 40° C. over a period of 1.5 h. Once the 40° C. target temperature is reached, about 0.5 volumes of water is added slowly to the crystallizer (above the surface) over a period of 5 min. to avoid any self-seeding, providing for a solvent ratio of about 96:4, DMSO:water (by volume). The solution is seeded by addition of 1% by weight of selpercatinib Form A seed crystals (D90 of —7 um). The seed crystals may be added as dry seeding crystals or as a slurry in a minimum volume of 80:20, DMSO:water (by volume). The seeded solution is incubated for about 30 min. After the 30 min. incubation, 2.5 volumes of room-temperature water is added over a period of 1 h. At the end of the water addition, the composition has a solvent ratio of about 80:20, DMSO:water (by volume).

Immediately after adding the 2.5 volumes of water, the reactor is cooled to 0° C. over a period of 2 h (rate of 20° C./h). Once at 0° C., the temperature of the slurry is maintained at 0° C. for 1 h. The solid is isolated by filtration, optionally at cooled temperature, at a rate that maintains a wet filter cake. The filtered solid is washed with 8 volumes of a first wash solution of DMSO/water (80/20, by volume) and the cake is filtered to dryness. The dry cake is washed with another 8 volumes of a second wash solution of water and filtered to dryness. To the dry cake is added another 8 volumes of water, with stirring (e.g., 30-60 s.) to re-suspend the solid cake material. The water washes are continued under the amount of residual DMSO detected in the sample is 0.5% or less. Once the residual DMSO threshold is reached, the filter cake is washed with 8 volumes of MTBE to displace water. An optional additional displacement wash using MTBE (8 volumes) may be performed to further reduce residual water content in the solid material. The resulting solid selpercatinib Form A is dried at 45° C. under vacuum, with a slight nitrogen gas flow maintained through the dryer. The resulting selpercatinib contains about 94 to about 98 wt % Form A.

### Example 2

### Gram-Scale Cooling Crystallization Process with Elevated Drying Temperatures to Produce Form A

Using a chemical synthesis reactor (Easymax, Mettler Toledo) approximately 6 g of selpercatinib is charged into a reactor along with (11 volumes) degassed DMSO and is heated at 70° C. under N₂ until the selpercatinib dissolves and the system reaches the 70° C. target temperature. The reactor was charged with additional DMSO (1 volume). The resulting solution is cooled to 40° C. over a period of 1.5 h. Once the 40° C. target temperature is reached 0.5 volumes of water is added slowly to the crystallizer (above the surface) over a period of 5 min. to avoid any self-seeding, providing for a solvent ratio of about 96:4, DMSO:water (by volume). The solution is seeded by addition of 1% by weight of selpercatinib Form A seed crystals. The seeded solution is incubated for about 30 min. After the 30 min. incubation, 2.5 volumes of room-temperature water is added over a period of 1 h. Immediately after adding the 2.5 volumes of water, the reactor is cooled to 0° C. over a period of 2 h. Once at 0° C., the temperature of the slurry is maintained at 0° C. for 1 h. The slurry is transferred from the reactor to a 10-micron disposable filter and fully de-liquored. The filtered solids are pulled under vacuum (e.g. 20 minutes). The filtered solid is then washed with 8 volumes of a first wash solution of DMSO/water (80/20, by volume) and the cake is filtered to dryness. The dry cake is washed with another 8 volumes of a second wash solution of water and filtered to dryness. To the dry cake is added 8 volumes of water, with stirring (e.g., 10-30 s.) to re-suspend the solid cake material. The solids are isolated by filtration. To the dry cake is added 8 volumes of MTBE, with stirring (e.g., 30 s.) to re-suspend the solid cake material. The solids are isolated by filtration. An optional additional displacement wash using MTBE may be performed to further reduce residual water content in the solid material. The resulting solid selpercatinib Form A is dried at 60° C. under vacuum, with a slight nitrogen gas flow maintained through the dryer.

Using the above methodology, a series of seven experiments were performed and summarized in Table 2, all under baseline conditions, to identify any baseline process variability. Two of the experiments were from batch on batch seeding experiments (032 and 033) and the experiments utilized different: starting material quality, Form B amount/quality in the seed crystal, and overall scale. The values in italics represent the HPLC integration of several known impurities that were in the starting material used in each experiment. The first set of impurity integrations for each line represents the impurity profile of the starting material, which the second set is the impurity profile of the isolated solids after crystallization.

TABLE 2
Summary of baseline process experiments

| | | | | DMSO | | | | | |
|---|---|---|---|---|---|---|---|---|---|
| | Selp. | | Resulting | wt. % | DMSO | | | COM- | N- |
| | starting | Seed added | Form B | (IPC-dry | final | KF | Amide ¹ | 1079² | Ethyl³ |
| Exp | material | (source) | (wt. %) | basis) | (wt. %) | (wt. %) | (A %) | (A %) | (A %) |
| 024 | A | 1 wt. % (a) | 2.2 | IPC1: 1.07 | 0.22 | 0.86 | 0.13 | 0.05 | 0.02 |
| | | | | IPC2: 0.28 | | | 0.06 | 0.01 | 0.03 |
| 025 | A | 1 wt. % (a) | 2.7 | 0.54 | 0.23 | 0.97 | 0.13 | 0.05 | 0.02 |
| | | | | 0.31 | | | 0.06 | ND | ND |
| 028 | A | 1 wt. % (a) | 2.7 | 0.31 | 0.21 | NR | 0.13 | 0.05 | 0.02 |
| | | | | NA | | | 0.06 | ND | ND |
| 029 | B | 1 wt. % (a) | 3.3 | 0.37 | 0.27 | 0.87 | 0.15 | 0.4 | 0.44 |
| | | | | NA | | | 0.1 | 0.17 | 0.21 |
| 032 | B | 1 wt. % (b) | <LOD | 0.49 | 0.36 | 0.72 | 0.15 | 0.4 | 0.44 |
| | | | | NA | | | 0.1 | 0.18 | 0.22 |
| 033 | B | 1 wt. % (b1) | <LOD | 0.49 | 0.43 | 0.73 | 0.15 | 0.4 | 0.44 |
| | | | | NA | | | 0.11 | 0.19 | 0.22 |
| 039 (10L) | C | 1 wt. % (a) | 5.7 | 0.72 | 0.20 | 0.46 | ND | 0.21 | ND |
| | | | | 0.28 | | | ND | 0.11 | ND |

¹ 4-[6-(3,6-diazabicyclo[3.1.1]heptan-3-yl)-3-pyridyl]-6-(2-hydroxy-2-methyl-propoxy)pyrazolo[1,5-a]pyridine-3-carboxamide.
²4-[6-(3,6-diazabicyclo[3.1.1]heptan-3-yl)-3-pyridyl]-6-(2-hydroxy-2-methyl-propoxy)pyrazolo[1,5-a]pyridine-3-carbonitrile.
³4-[6-(6-ethyl-3,6-diazabicyclo[3.1.1]heptan-3-yl)-3-pyridyl]-6-(2-hydroxy-2-methyl-propoxy)pyrazolo[1,5-a]pyridine-3-carbonitrile.
(a) Seed was a single lot that contained 1.6% Form B and had a d90 of 6 um.
(b) Seed was a single lot that contained 3.3% Form B and had a d90 of 29 um.
(b1) Seed was a single lot but contained ND Form B and had a d90 of 67 um.
A. The starting material was a relatively clean batch
B. The starting material was a batch with impurities for testing impurity rejection.
C. The starting material was a second relatively clean batch.

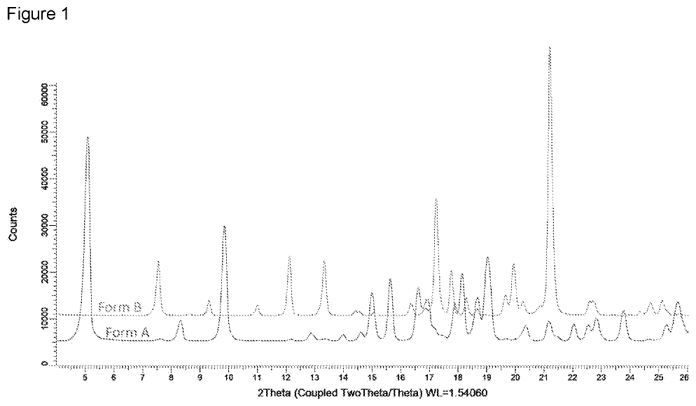

The HPLC method used for analysis of the final solids is given in Table 3 and an example chromatogram shown in FIG. 1. COM-1074 is 6-methoxy nicotinaldehyde.

TABLE 3
Development HPLC method for crystallization development.

| Column | Waters XBridge Shield C18 (4.6 mm × 75 mm, | | |
|---|---|---|---|
| | 3.5 μm). | | |
| Mobile phase A | 0.1% TFA in water | | |
| Mobile phase B | 0.1% TFA in ACN | | |
| Column Temperature | 25° C. | | |
| Flow rate | 0.7 ml/min | | |
| Gradient profile | Time | % A | % B |
| | 0 | 82 | 18 |
| | 5.25 | 15 | 85 |
| | 5.5 | 82 | 18 |
| | 7 | 82 | 18 |

Seed crystal of sufficient quality of selpercatinib Form A can enhance growth and secondary nucleation of the desired form and to reduce variability from an unseeded process that relies on, e.g., primary nucleation. Seed crystal specifications can be used to control the amount of allowable Form B content in the seed.

### Example 3

### Reverse Addition Process for Direct Isolation of Form A

DMSO was saturated with excess form B at RT. From this slurry, the liquors were obtained via filtration. 25 ml of the saturated DMSO solution was taken up into a syringe and were charged at 1 ml/min to a pot containing 15 ml of water at 20° C. (˜63/37 DMSO/H2O). Immediate crystallization was observed throughout the addition. At the end of the addition, a sample of the solids was taken and found to have non-detect Form B via XRPD analysis. Alternative DMSO and water volumes (and thus DMSO/H2O ratio) is expected to have similar control over Form B due to the high driving force. Ratios ranging from 90/10 to 20/80 are expected to give similar performance.

### Example 4

### Co-Addition Process for Direct Isolation of Form A

Experiments demonstrated a co-addition designed to maintain a solvent composition of either 80/20 volume% or 90/10 volume% DMSO/water in the crystallization mixture by using a pure selpercatinib/DMSO feed stream and a water feed stream added simultaneously to a pot containing a seed bed in the corresponding DMSO/water solvent system. An example process description for the 80/20 process is given below.

Prepare water (antisolvent) feed by drawing up 3 volumes into a syringe.

Prepare the API feed by dissolving 1 equivalent basis of the API, which could be Form A or Form B, into 12 volumes of DMSO and heat to 65° C. to obtain a solution. This solution was taken up into a syringe for dispensing. To prevent crystallization, this feed should be maintained hot, however for short timescales on the order of hours, it can be allowed to cool to RT without crystallization.

Prepare the crystallizer pot by charging 3.2 volumes of DMSO, 0.8 volumes of water (targeting 4 volumes of 80/20 ratio to be suitable volume for agitation) and equilibrate to 20° C. Charge 1 wt % (optional) Form A seed and start stirring.

Next start the co-addition by feeding both feeds over 4 hours, the volumetric flow rates and volumes are designed to maintain the 80/20 DMSO/water ratio constant.

After the co-addition the slurry can be isolated immediately or after extended hold.

Using the above methodology, a series of eight experiments are performed and summarized in Table 4, to identify significant factors to Form purity. Several of the experiments utilized a mixture of Form A and Form B seeds to test the robustness of the conditions.

TABLE 4
Summary of conditions to identify factors to Form purity

| | | Selp. | | | | | | | COM- | N- |
|---|---|---|---|---|---|---|---|---|---|---|
| | | starting | Stirring | Seed added | | | Form B | Amide ¹ | 1079² | Ethyl³ |
| Exp | Process | material | rpm | (source) | | Isolation | (wt %) | (A %) | (A %) | (A %) |
| 1 | 80/20 | B | 250 | 1 | wt % (c) | H₂O, MeOH, MTBE | 1 | 0.15 | 0.4 | 0.44 |
| | | | | | | displacement washes | | 0.16 | 0.34 | 0.37 |
| 2 | 80/20 | A | 250 | 1 | wt % (d) | H₂O, MeOH, MTBE | ND | — | — | — |
| | | | | | | displacement washes | | | | |
| 3 | 80/20 | A | 1000 | 1 | wt % (d) | H₂O, MeOH, MTBE | 1.4 | — | — | — |
| | | | | | | displacement washes | | | | |
| 4 | 80/20 | A | 600 | 1 | wt % (d) | H₂O, MeOH, MTBE | ND | — | — | — |
| | | | | | | displacement washes | | | | |
| 5 | 80/20 | B | 300 | 10 | wt % (e) | H₂O, MeOH, MTBE | 6.3 | 0.15 | 0.4 | 0.44 |
| | | | | | | displacement washes | | 0.17 | 0.33 | 0.35 |
| 6 | 90/10 | B | 300 | 2.5 | wt % (f) | H₂O, MeOH, MTBE | 5 | 0.15 | 0.4 | 0.44 |
| | | | | | | displacement washes | | 0.14 | 0.23 | 0.25 |
| 7 | 90/10 | B | 300 | | | 70/30 DMSO/H₂O, | 5.7 | 0.15 | 0.4 | 0.44 |
| | | | | 2.1 | wt % (d) | H₂O, MeOH (all | | 0.13 | 0.25 | 0.31 |
| | | | | | | reslurry) | | | | |
| 8 | 90/10 | B | 800 | 2.1 | wt % (d) | 70/30 DMSO/H₂O, | 8.8 | 0.15 | 0.4 | 0.44 |
| | | | | | | H₂O, MeOH (all | | 0.12 | 0.24 | 0.30 |
| | | | | | | reslurry) | | | | |

(c) 90/10 ratio of Form A and Form B was used.
(d) 90/10 ratio of Form A and Form B was used but with a different lot of Form B compared to (c).
(e) A single lot of Form A which contained 1.6% Form B.
(f) 95/5 ratio of Form A and Form B was used using the same lots as in (d).
¹4-[6-(3,6-diazabicyclo[3.1.1]heptan-3-yl)-3-pyridyl]-6-(2-hydroxy-2-methyl-propoxy)pyrazolo[1,5-a]pyridine-3-carboxamide.
²4-[6-(3,6-diazabicyclo[3.1.1]heptan-3-yl)-3-pyridyl]-6-(2-hydroxy-2-methyl-propoxy)pyrazolo[1,5-a]pyridine-3-carbonitrile.
³4-[6-(6-ethyl-3,6-diazabicyclo[3.1.1]heptan-3-yl)-3-pyridyl]-6-(2-hydroxy-2-methyl-propoxy)pyrazolo[1,5-a]pyridine-3-carbonitrile.
A. The starting material was a relatively clean batch
B. The starting material was a batch with impurities for testing impurity rejection.

The results showed that 80/20 conditions provided better Form A control than the 90/10 conditions due to the higher supersaturation level. Thus higher percentage water/DMSO ratios such as 50/50 or 20/80 can also be used to ensure high Form A purity. The demonstrated co-addition conditions are also representative of a continuous crystallization process where continuous feeding and removal of slurry could be performed.

### Example 5

### Solvate Preparation and Conversion Process

Selpercatinib can form solvates with solvent molecules, the majority of which are not stable upon drying. In this example, Form A selpercatinib is prepared from a dichloromethane (DCM) solvate.

In a reaction vessel, selpercatinib (0.8751 g, API) and water-saturated DCM (29.55 vols) are mixed and heated (35° C.) to dissolution. As an alternative, the same volume of DCM without water saturation can be used as solvent to achieve similar results. Once the selpercatinib is dissolved, heptane is added (10 vols) over 30 min. After the addition of heptane is completed, the mixture is cooled to a target temperature of 25° C., over 30 min. Once the target temperature is reached, a second tranche of heptane (10 vols) is added to the mixture over 30 min. After the addition of the second tranche of heptane is complete, the mixture is stirred for at least 8 h. at ambient temperature (25° C.). The resulting solid is isolated and washed (one wash with 4 vol heptane, a second wash with 4 vol MTBE), and dried at 45° C.

The resulting solid produced by this process is characterized as a DCM solvate that forms at the end of the crystallization, and which converts to Form A upon drying. The formation of the solvate appears to remove any dependence on, or effect from, the seed crystal form.

Disclosed herein is a compound of Formula I, wherein the compound of Formula I contains at least about 90 wt % of Form A and wherein the compound of Formula I is obtained by adding selpercatinib to DMSO to form a mixture, heating the mixture to about 50-70° C. to dissolve the selpercatinib and thereby form a solution, cooling the solution to about 40° C. and then adding a first batch and a second batch of water. The first batch of water may be e.g., about 0.5 volumes of water, optionally seeding the selpercatinib/DMSO/water mixture with seed crystals, adding a second batch of water of about 2.5 volumes of water, then cooling the mixture to about 0° C., and isolating the selpercatinib Form A. After the addition of the first batch of water, the ratio of the DMSO:water is about 96:4. After the addition of the second batch of water, e.g., 2.5 volumes of water, the ratio of the DMSO:water is about 80:20. The isolated Form A is washed with about 8 volumes of DMSO:water (80:20), filtered to dryness, washed a second time with another 8 volumes of DMSO:water (80:20), and again filtered to dryness. The cake is then suspended in about 8 volumes of water and filtered. This process is repeated until the amount of residual DMSO detected in the sample is 0.5% or less. The filter cake is then washed at least once with about 8 volumes of MTBE. The selpercatinib Form A is then dried under vacuum, at a temperature of about 45° C.

Embodiments

Embodiment 1. A method of converting selpercatinib to selpercatinib Form A comprising:
a) dissolving selpercatinib in a solvent comprising DMSO and thereby forming a selpercatinib DMSO solution;
b) adding water to the selpercatinib DMSO solution to form a slurry; and
c) isolating the crystallized selpercatinib Form A from the slurry, wherein the Form A has XRPD peaks at about 4.9, 9.7, and 15.5°2θ; or
d) dissolving the selpercatinib in a solvent comprising dichloromethane to form a solution;
e) adding heptane to the solution and under conditions effective to form a slurry;
f) isolating the selpercatinib Form A from the slurry, wherein the Form A has XRPD peaks at about 4.9, 9.7, and 15.5°2θ

Embodiment 2. A method for converting selpercatinib to selpercatinib Form A, the method comprising:
a) dissolving selpercatinib in a solvent comprising DMSO and thereby forming a selpercatinib DMSO solution;
b) adding water to the selpercatinib DMSO solution to form a slurry; and
c) isolating the crystallized selpercatinib Form A from the slurry, wherein the Form A has XRPD peaks at about 4.9, 9.7, and 15.5°2θ.

Embodiment 3. The method according to embodiment 2, wherein about 1 gram of selpercatinib is dissolved in about 10-15 mL of DMSO.

Embodiment 4. The method according to embodiment 2 or 3, wherein step a comprises heating the DMSO and selpercatinib to a temperature of about 50 to 70° C.

Embodiment 5. The method according to any one of embodiment 2-4, wherein step b comprises adding a first batch of water and a second batch of water.

Embodiment 6. The method according to embodiment 5, wherein after the first batch of water is added, the ratio of DMSO to water is about 96:4 by volume.

Embodiment 7. The method according to any one of embodiments 5-6 comprising, cooling the DMSO and selpercatinib to about 40° C. before the first batch of water is added.

Embodiment 8. The method according to any one of embodiments 5-7, wherein after the second batch of water is added, the ratio of DMSO:water is about 80:20.

Embodiment 9. The method according to any one of embodiments 5-8, comprising adding the second batch of water and cooling the DMSO:water to about 0° C., and thereby forming a slurry.

Embodiment 10. The method according to any one of embodiments 2-9, wherein step b comprises adding about 0.1 to about 1 mL/g of water to the solution.

Embodiment 11. The method according to any one of embodiments 2-10, wherein step b comprises adding about no more than about 0.2 mL/g of water to the solution.

Embodiment 12. The method according to any one of embodiments 2-11, further comprising adding selpercatinib seed crystals to the DMSO:water.

Embodiment 13. The method according to embodiment 12, wherein about 1 to 15 wt % of selpercatinib Form A seed crystals is added to the DMSO:water.

Embodiment 14. The method according to embodiments 12 or 13, wherein about 1 wt % of selpercatinib Form A seed crystals is added to the DMSO:water.

Embodiment 15. The method according to any one of embodiments 12-14, comprising adding the selpercatinib seed crystals before adding the second batch of water.

Embodiment 16. The method according to any one of embodiment 2-15, wherein step c comprises vacuum filtration.

Embodiment 17. The method according to any one of embodiments 2-15, wherein step c comprises centrifugal separation.

Embodiment 18. The method according to any one of embodiments 2-17, comprising washing the isolated selpercatinib Form A from step c with a solvent comprising MTBE and/or water.

Embodiment 19. The method according to any one of embodiments 2-18, further comprising drying the selpercatinib Form A.

Embodiment 20. A method for converting selpercatinib to selpercatinib Form A, the method comprising:
a. dissolving the selpercatinib in a solvent comprising dichloromethane to form a solution;
b. adding heptane to the solution and under conditions effective to form a slurry;
c. isolating the selpercatinib Form A from the slurry, wherein the Form A has XRPD peaks at about 4.9, 9.7, and 15.5°2θ.

Embodiment 21. The method according to embodiment 20, wherein about 1 gram of selpercatinib is dissolved in about 25-35 mL of dichloromethane.

Embodiment 22. The method according to any one of embodiments 20-21, wherein step a comprises heating the selpercatinib and the solvent comprising dichloromethane to about 30° C. to 40° C.

Embodiment 23. The method according to any one of embodiments 20-22, wherein step b comprises adding a first batch of heptane and a second batch of heptane.

Embodiment 24. The method according to embodiment 23, wherein the first batch of heptane comprises about 8-12 mL of heptane/g of selpercatinib.

Embodiment 25. The method according to embodiments 23 or 24, wherein the second batch of heptane comprises about 8-12 mL of heptane/g of selpercatinib.

Embodiment 26. The method according to any of embodiments 20-25, wherein step b comprises cooling to a temperature of less than about 30° C. and greater than about 20° C.

Embodiment 27. The method according to embodiment 26, wherein step b comprises cooling to a temperature of about 25° C.

Embodiment 28. The method according to any one of embodiments 20-27, wherein step b comprises stirring for at least about 8 h.

Embodiment 29. A pharmaceutical composition comprising selpercatinib Form A made according to any of embodiments 1-28.

Embodiment 30. The composition according to embodiment 29, further comprising at least one pharmaceutically acceptable carrier, diluent, or excipient.

Embodiment 31. The pharmaceutical composition according to embodiment 29 or 30, wherein the composition contains less than about 20% by wt. of other crystal forms of selpercatinib.

Embodiment 32. The pharmaceutical composition according to embodiment 29 or 30, wherein the composition contains less than about 10% by wt. of other crystal forms of selpercatinib.

Embodiment 33. The pharmaceutical composition according to embodiment 29 or 30, wherein the composition contains less than about 5% by wt. of other crystal forms of selpercatinib.

Embodiment 34. The pharmaceutical composition according to embodiment 29 or 30, wherein the composition comprising selpercatinib Form A is substantially pure.

Embodiment 35. A method of treating cancer in a patient comprising administering to a patient in need of such treatment an effective amount of selpercatinib Form A made according to any of embodiments 1-28 or a pharmaceutical composition according to any of claims 29-34.

Embodiment 36. The method of embodiment 35, wherein the cancer is a RET associated cancer.

Embodiment 37. The method of embodiment 35 or 36, wherein the cancer selected from the group consisting of: solid tumors, lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer.

Embodiment 38. The method according to embodiment 37, wherein the cancer is medullary thyroid cancer.

Embodiment 39. The method according to embodiment 37, wherein the cancer is lung cancer and the lung cancer is small cell lung carcinoma, non-small cell lung cancer, bronchioles lung cell carcinoma, RET fusion lung cancer, or lung adenocarcinoma.

Embodiment 40. The method according to embodiment 37, wherein the cancer is solid tumors.

Embodiment 41. The method according to embodiment 37 or 40, wherein the solid tumors are locally advanced or metastatic solid tumors.

Embodiment 42. The method according to embodiment 41, wherein the solid tumors are locally advanced or metastatic solid tumors with a RET gene fusion that have progressed on or following prior systemic treatment or who have no satisfactory alternative treatment options.

Embodiment 43. The method according to embodiment 35 or 36, wherein the cancer is locally advanced or metastatic non-small cell lung cancer (NSCLC) with a rearranged during transfection (RET) gene fusion, as detected by an FDA-approved test.

Embodiment 44. The method according to embodiment 35 or 36, wherein the cancer is advanced or metastatic thyroid cancer with a RET gene fusion, as detected by an FDA-approved test, who require systemic therapy and who are radioactive iodine-refractory (if radioactive iodine is appropriate).

Embodiment 45. The method according to any one of embodiments 35-44, wherein the pharmaceutical composition contains about 40 mg of selpercatinib Form A.

Embodiment 46. The method according to any one of embodiments 35-44, wherein the pharmaceutical composition contains about 80 mg of selpercatinib Form A.

Embodiment 47. The method according to any one of embodiments 35-44, wherein the pharmaceutical composition contains about 120 mg of selpercatinib Form A.

Embodiment 48. The method according to any one of embodiments 35-44, wherein the pharmaceutical composition contains about 160 mg of selpercatinib Form A.

Embodiment 49. The method according to any one of embodiments 35-48, wherein the pharmaceutical composition is provided in a tablet.

Embodiment 50. The method according to any one of embodiments 35-48, wherein the pharmaceutical composition is provided in a capsule.

Embodiment 51. A pharmaceutical composition comprising at least about 80 wt % selpercatinib Form A or a pharmaceutically acceptable salt thereof, for use in therapy, wherein the pharmaceutical composition comprises selpercatinib Form A that was made according to any one of embodiments 1-50.

Embodiment 52, The pharmaceutical composition comprising at least about 80 wt % selpercatinib Form A or a pharmaceutically acceptable salt thereof, for use according to embodiment 51, further comprising at least one pharmaceutically acceptable carrier, diluent, or excipient.

Embodiment 53. The pharmaceutical composition for use according to embodiment 51 or 52, wherein the pharmaceutical composition contains less than about 20% by wt. of other forms of selpercatinib.

Embodiment 54. The pharmaceutical composition for use according to embodiment 51 or 52, wherein the composition contains less than about 10% by wt. of other forms of selpercatinib.

Embodiment 55. The pharmaceutical composition for use according to embodiment 51 or 52, wherein the composition contains less than about 5% by wt. of other forms of selpercatinib.

Embodiment 56. The pharmaceutical composition for use according to embodiment 51 or 52, wherein the composition comprising selpercatinib Form A is substantially pure.

Embodiment 57. A pharmaceutical composition comprising at least about 80 wt % selpercatinib Form A or a pharmaceutically acceptable salt thereof, for use in treating cancer.

Embodiment 58. A pharmaceutical composition comprising at least about 80 wt % selpercatinib Form A or a pharmaceutically acceptable salt thereof, for use in treating cancer, wherein the pharmaceutical composition comprises selpercatinib Form A that was made according to any one of embodiments 1-50.

Embodiment 59. The pharmaceutical composition for use according to embodiment 57 or 58, wherein the pharmaceutical composition contains less than about 20% by wt. of other forms of selpercatinib.

Embodiment 60. The pharmaceutical composition for use according to embodiment 57 or 58, wherein the composition contains less than about 10% by wt. of other forms of selpercatinib.

Embodiment 61. The pharmaceutical composition for use according to embodiment 57 or 58, wherein the composition contains less than about 5% by wt. of other forms of selpercatinib.

Embodiment 62. The pharmaceutical composition for use according to any one of embodiments 57-61, wherein the cancer is a RET-associated cancer.

Embodiment 63. The pharmaceutical composition for use according to any one of embodiments 57-61, the cancer is selected from the group consisting of: solid tumors, lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer.

Embodiment 64. The pharmaceutical composition for use according to embodiment 63, wherein the cancer is medullary thyroid cancer.

Embodiment 65. The pharmaceutical composition for use according to embodiment 63, wherein the cancer is lung cancer and the lung cancer is small cell lung carcinoma, non-small cell lung cancer, bronchioles lung cell carcinoma, RET fusion lung cancer, or lung adenocarcinoma.

Embodiment 66. The pharmaceutical composition for use according to embodiment 62 or 63, wherein the cancer is RET fusion lung cancer.

Embodiment 67. The pharmaceutical composition for use according to embodiment 63, wherein the cancer is solid tumors.

Embodiment 68. The pharmaceutical composition for use according to embodiment 63 or 67, wherein the solid tumors are locally advanced or metastatic solid tumors.

Embodiment 69. The pharmaceutical composition for use according to embodiment 63, 67, or 68, wherein the solid tumors are locally advanced or metastatic solid tumors with a RET gene fusion that have progressed on or following prior systemic treatment or who have no satisfactory alternative treatment options.

Embodiment 70. The pharmaceutical composition for use according to embodiment 63, wherein the cancer is locally advanced or metastatic non-small cell lung cancer (NSCLC) with a rearranged during transfection (RET) gene fusion, as detected by an FDA-approved test.

Embodiment 71. The pharmaceutical composition for use according to embodiment 63, wherein the cancer is advanced or metastatic thyroid cancer with a RET gene fusion, as detected by an FDA-approved test, who require systemic therapy and who are radioactive iodine-refractory (if radioactive iodine is appropriate).

Embodiment 72. The pharmaceutical composition for use according to any one of embodiments 51-71, wherein the pharmaceutical composition contains about 40 mg of selpercatinib Form A.

Embodiment 73. The pharmaceutical composition for use according to any one of embodiments 51-71, wherein the pharmaceutical composition contains about 80 mg of selpercatinib Form A.

Embodiment 74. The pharmaceutical composition for use according to any one of embodiments 51-71, wherein the pharmaceutical composition contains about 120 mg of selpercatinib Form A.

Embodiment 75. The pharmaceutical composition for use according to any one of embodiments 51-71, wherein the pharmaceutical composition contains about 160 mg of selpercatinib Form A.

Embodiment 76. The pharmaceutical composition for use according to any one of embodiments 51-75, wherein the pharmaceutical composition is provided in a tablet.

Embodiment 77. The pharmaceutical composition for use according to any one of embodiments 51-75, wherein the pharmaceutical composition is provided in a capsule.

What is claimed is:

1. A method for converting selpercatinib to selpercatinib Form A, the method comprising:
a. dissolving selpercatinib in a solvent comprising DMSO and thereby forming a selpercatinib DMSO solution;
b. adding water to the selpercatinib DMSO solution to form a slurry; and
c. isolating the crystallized selpercatinib Form A from the slurry, wherein the Form A has XRPD peaks at about 4.9, 9.7, and 15.5°2θ.

2. The method according to claim 1, wherein about 1 gram of selpercatinib is dissolved in about 10-15 mL of DMSO.

3. The method according to claim 1, wherein step a comprises heating the DMSO and selpercatinib to a temperature of about 50 to 70° C.

4. The method according to claim 1, wherein step b comprises adding a first batch of water and a second batch of water.

5. The method according to claim 4, wherein after the first batch of water is added, the ratio of DMSO to water is about 96:4 by volume.

6. The method according to claim 4 comprising, cooling the DMSO and selpercatinib to about 40° C. before the first batch of water is added.

7. The method according to claim 4, wherein after the second batch of water is added, the ratio of DMSO:water is about 80:20.

8. The method according to claim 4, comprising adding the second batch of water and cooling the DMSO:water to about 0° C., and thereby forming a slurry.

9. The method according to claim 1, wherein step b comprises adding about 0.1 to about 1 mL/g of water to the solution.

10. The method according to claim 9, wherein step b comprises adding about no more than about 0.2 mL/g of water to the solution.

11. The method according to claim 1, further comprising adding selpercatinib seed crystals to the DMSO:water.

12. The method according to claim 11, wherein about 1 to 15 wt % of selpercatinib Form A seed crystals is added to the DMSO:water.

13. The method according to claim 11, wherein about 1 wt % of selpercatinib Form A seed crystals is added to the DMSO:water.

14. The method according to claim 11, comprising adding the selpercatinib seed crystals before adding the second batch of water.

15. The method according to claim 1, wherein step c comprises vacuum filtration or centrifugal separation.

16. The method according to claim 15, comprising washing the isolated selpercatinib Form A from step c with a solvent comprising MTBE and/or water.

17. The method according to claim 2, wherein step a comprises heating the DMSO and selpercatinib to a temperature of about 50 to 70° C.; and step b comprises adding a first batch of water and a second batch of water and cooling the DMSO and selpercatinib to about 40° C. before the first batch of water is added.

18. A method for converting selpercatinib to selpercatinib Form A, the method comprising:
a. dissolving the selpercatinib in a solvent comprising dichloromethane to form a solution;
b. adding heptane to the solution and under conditions effective to form a slurry;
c. isolating the selpercatinib Form A from the slurry, wherein the Form A has XRPD peaks at about 4.9, 9.7, and 15.5°2θ.

19. The method according to claim 18, wherein about 1 gram of selpercatinib is dissolved in about 25-35 mL of dichloromethane.

20. The method according to claim 18, wherein step a comprises heating the selpercatinib and the solvent comprising dichloromethane to about 30° C. to 40° C.

21. The method according to claim 18, wherein step b comprises adding a first batch of heptane and a second batch of heptane.

22. The method according to claim 21, wherein the first batch of heptane comprises about 8-12 mL of heptane/g of selpercatinib.

23. The method according to claim 21, wherein the second batch of heptane comprises about 8-12 mL of heptane/g of selpercatinib.

24. The method according to claim 18, wherein step b comprises cooling to a temperature of less than about 30° C. and greater than about 20° C.

25. The method according to claim 18, wherein step b comprises stirring for at least about 8 h.

26. The method according to claim 19, wherein step a comprises heating the selpercatinib and the solvent comprising dichloromethane to about 30° C. to 40° C.; step b comprises adding a first batch of heptane and a second batch of heptane, wherein the first batch of heptane comprises about 8-12 mL of heptane/g of selpercatinib; wherein the second batch of heptane comprises about 8-12 mL of heptane/g of selpercatinib; and wherein step b comprises cooling to a temperature of less than about 30° C. and greater than about 20° C.

27. A pharmaceutical composition comprising selpercatinib Form A made according to claim 1.

28. The composition according to claim 27, further comprising at least one pharmaceutically acceptable carrier, diluent, or excipient.

29. The pharmaceutical composition according to claim 28, wherein the composition contains less than about 20% by wt. of other crystal forms of selpercatinib.

30. The pharmaceutical composition according to claim 28, wherein the composition comprising selpercatinib Form A is substantially pure.

31. A method of treating cancer in a patient comprising administering to a patient in need of such treatment an effective amount of selpercatinib Form A made according to claim 1.

32. The method of claim 31, wherein the cancer is a RET associated cancer.

33. The method of claim 32, wherein the cancer selected from the group consisting of: solid tumors, lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer.

34. The method according to claim 33, wherein the cancer is medullary thyroid cancer.

35. The method according to claim 33, wherein the cancer is lung cancer and the lung cancer is small cell lung carcinoma, non-small cell lung cancer, bronchioles lung cell carcinoma, RET fusion lung cancer, or lung adenocarcinoma.

36. The method according to claim 33, wherein the cancer is solid tumors.

37. The method according to claim 33, wherein the solid tumors are locally advanced or metastatic solid tumors.

38. The method according to claim 36, wherein the solid tumors are locally advanced or metastatic solid tumors with a RET gene fusion that have progressed on or following prior systemic treatment or who have no satisfactory alternative treatment options.

39. The method according to claim 32, wherein the cancer is locally advanced or metastatic non-small cell lung cancer (NSCLC) with a rearranged during transfection (RET) gene fusion, as detected by an FDA-approved test.

40. The method according to claim 32, wherein the cancer is advanced or metastatic thyroid cancer with a RET gene fusion, as detected by an FDA-approved test, who require systemic therapy and who are radioactive iodine-refractory (if radioactive iodine is appropriate).
